(19) United States
(12) Patent Application Publication
(10) Pub. No.: US 2011/0319802 A1
(43) Pub. Date: Dec. 29, 2011

(54) FOOT CORRECTION DEVICE

(76) Inventor: Mauro Luciano NARDINI, Buenos Aires (AR)

(21) Appl. No.: 13/168,038

(22) Filed: Jun. 24, 2011

(30) Foreign Application Priority Data
Jun. 25, 2010 (AR) 20100102259

(51) Int. Cl. A61F 5/00

(52) U.S. Cl. 602/28

(57) ABSTRACT

A foot correction device to aid and improve the step pressure points in a humans foot, wherein the device comprises a first element containing a plurality of separating means, wherein said first element is connected to a second element comprising fixing and adjusting means of said foot correction device to the user's foot, having said second element a plurality of backing supplements. A method to reeducate the global posture of a human's foot using a foot correction device, the method comprises the steps of inserting the toes between the gaps defined in the separating means; sliding the device until the separating means tops with the toe's joints; adjusting the separating means using the adjustable means; proceeding with the adjustment by means of the fixing and adjusting means of the second element and optionally performing several specific routines

### TECHNICAL FIELD

The present invention relates to a foot correction device, which corrects the pressure points of a standing person. The invention is also related to a method re-educating the global positioning of a standing person using said device.

### BACKGROUND

It is known to a person skilled in the art that one of the most complex parts in the human body are actually the feet. The human foot and ankle is a strong and complex mechanical structure containing more than 26 bones, 33 joints (20 of which are actively articulated), and more than a hundred muscles, tendons, and ligaments. The human foot is prone to suffer a huge amount of stress (and bad postures). As mentioned before the 33 joints, eight arches, 26 bones, more than a hundred muscles, ligaments, and tendons they all work together to distribute body weight and allow movement. Unfortunately, many people pay no attention to their feet—until they start to hurt. Foot disorders must be diagnosed and treated early, before they become very painful and incapacitating. In some cases, some painful foot abnormalities are already warning signs of even more serious ailments such as diabetes, circulatory disorders, and nerve problems.

Anybody who has ever worn uncomfortable shoes has probably had foot corns and calluses problems. They turn up when too much pressure is applied on the foot's skin, such as when the bones of the feet press against tight shoes and also press against each other. Most people get foot corns and calluses on the big toes, fifth toes, and metatarsals, where pressure is particularly rougher. Fortunately, this type of foot condition is fairly easy to treat. Doctors will prescribe wider shoes or cushion pads to relieve pressure. In some advanced cases though, surgery may be needed.

As a common solution to several feet condition, there are many implementations which bring relief to such pain improving the person's posture. However, some of those solutions are not totally effective.

One of the most commonly known devices are the ones available under the brand PAMPERED TOES. Such a device stretches and relaxes the person's toes allowing some passive movement of the foot. Another device which includes similar characteristics is the Night Time Bunion Corrector. A bunion night splint can be used to reduce the need to use surgical intervention for a bunion joint. This product is used to correct bunions while the person is sleeping.

Furthermore, is it also known the U.S. Pat. No. 5,076,256 to Funatogawa, Hiroyuki, This patent teaches a toe stretcher comprising stretching posts stood upright at positions corresponding to spacings between toes, the stretching posts being formed of an elastic material having a diameter capable of spreading a portion between toes on a base plate of the size capable of putting the tip of toes thereon.

Also, U.S. Pat. No. 7,322,915 in the name of Ferri Federic, teaches an exercise tool specifically devised as a toe stretcher to align, separate, and stretch toes. The toe stretcher comprises a frame with a separator for separating a plurality of toes. The frame further includes holes or indentations for placement of the toes. Additionally, the frame is flexible, allowing a user to easily manipulate the toe stretcher and place it on the user's toes, effectively separating and stretching toes. Furthermore, the toe stretcher may be attached with footwear, allowing a user to not only stretch toes, but to also use the toe stretcher as footwear.

Even though the devices aforementioned provide a solution to the problems commonly encounter in a person's feet, none of them teaches the features provided in the subject matter of the present invention.

### SUMMARY OF THE INVENTION

The present invention relates to a foot correction device, which corrects the pressure points of a standing person. The invention is also related to a method re-educating the global positioning of a standing person using said device.

In another aspect, the device corrects the several pressure points when a person stands up, being the device applied in a person's foot, avoiding the stress in the articulations and toes during its use.

Furthermore, the device is anatomically configured and provides the user with an ease of use.

In yet another aspect, the device facilitates the stretching of the toes and at the same time corrects the position of the person.

Additionally, the present invention provides a foot correction device which corrects the pressure points in the footsteps of a person, wherein the device comprises a first element comprising a plurality of separating means, wherein the first element is connected to a second element which also comprises fixing and adjusting means of said device onto the user's foot, having said second element a plurality of backing supplements.

It is also an object of the invention to provide a method for correcting the positioning of a person's foot using the foot correction device, wherein the method comprises the step of inserting the toes between the space defined between the separating means, sliding the correcting device until the separator means reach the joint of the toes, adjusting the separating means with the fixing and adjusting means of the second element and optionally perform several specific routines.

### BRIEF DESCRIPTION OF THE FIGURES

The skilled artisan will understand that the drawings, described below, are for illustration purposes only. The drawings are not intended to limit the scope of the present teachings in any way.

FIG. 1 is a perspective view from the top of the device of the present invention;

FIG. 2 is a perspective view from the bottom of the first element of the device of FIG. 1;

FIG. 3 is a top view showing the use of the device of the present invention of FIG. 1; and

FIG. 4 illustrates in further details one of the adjusting means to be used in the device of FIG. 1.

### DESCRIPTION OF THE PREFERRED EMBODIMENT

As used throughout the present disclosure, the technical and scientific terms used in the descriptions herein will have the meanings commonly understood by one of ordinary skill in the art, unless specifically defined otherwise.

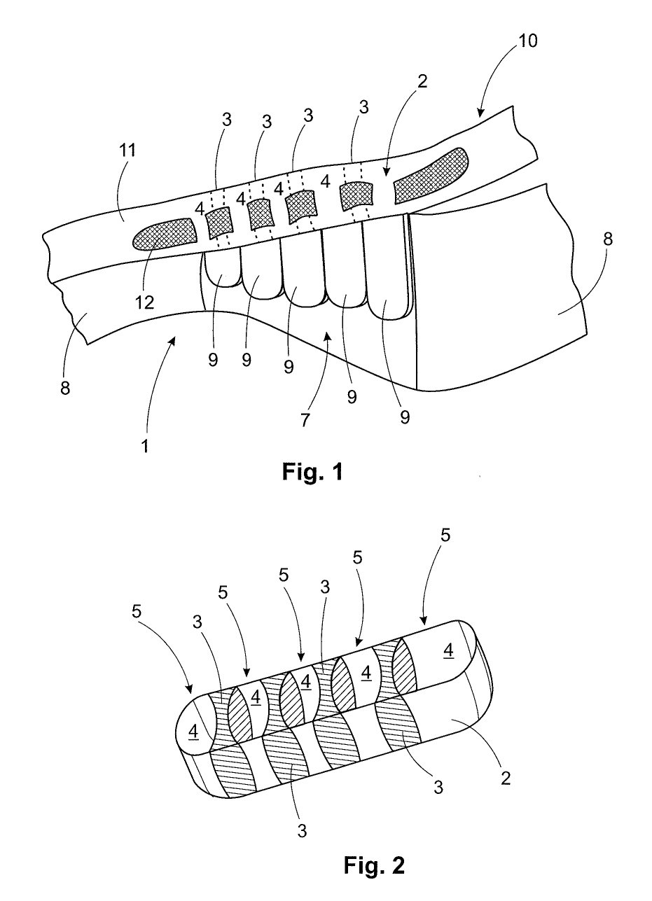

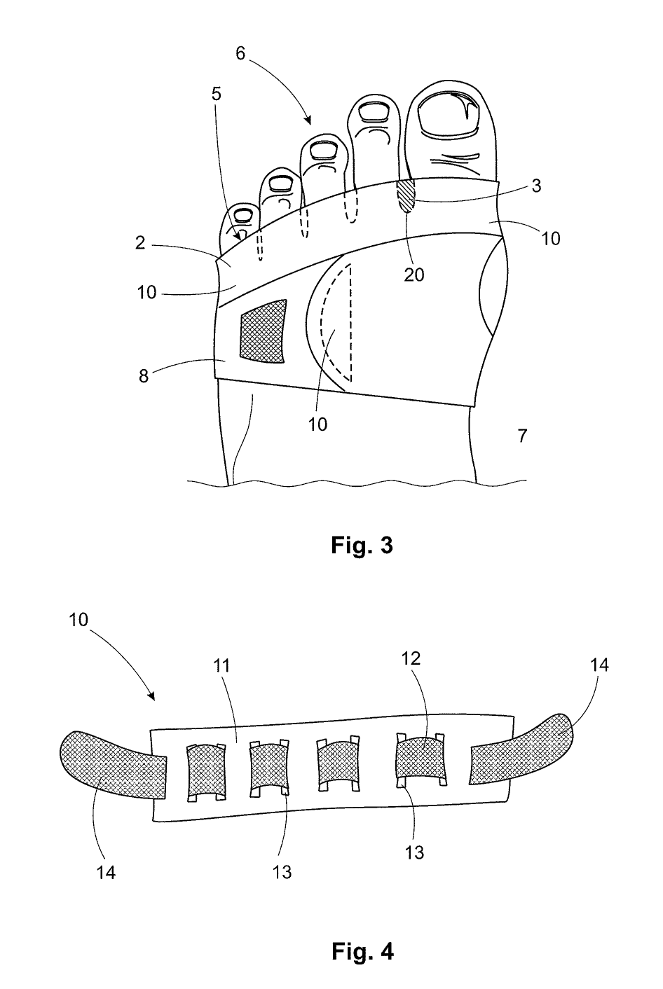

In referring specifically to the drawings, and in particular FIG. 1, this sketch shows the foot correction device 1 comprising a first element 2 which in turn comprises a plurality of separating means 3. Said separating means 3 presents a gap 4 between each of them defining a path 5 for the toes 6 (As illustrated in FIG. 2).

The first element 2 y connected to a second element 7 which comprises fixing and adjusting means 8. The fixing and adjusting means 8 are used to fix and adjust the device 1 to the foot 6 of the user. As shown in FIG. 1, the second element 7 presents a plurality of backing elements or supplements 9. On the other hand, the first element 2 comprises adjustable means 10, which allows regulating the distance between said separating means 3 for the correct fit to the user's foot 6. In this particular embodiment the variable adjusting means 10 are made from a main fabric 11 and a secondary fabric 12.

As shown in FIG. 1, but in more detail in FIG. 4, the secondary fabric 12 is threaded with the main fabric 11, passing through a sliding rib 13. This sliding rib 13 is placed between the separating means 3. It is important to mention that the device 1 can contain one or more of those variable adjustable means 10. In the event that the device is configured to include at least two adjustable means 10, the same can be fastened each other by the ends 14 in order to obtain a correct fixation of the device in the user's foot.

In addition, the main fabric 11 and the secondary fabric 12 are independent from each other and both allow adjusting the separating means 3 towards the toes without the use of excessive force. This particular embodiment avoids the creation of bends or folds that may result from the adjusting of both fabrics 11 and 12.

Given that the device 1 subject of the present invention included several applications depending in its use, the backing supplements 9 can be placed on the top or the bottom portion of the second element. However, the backing supplements 9 should always be placed in the inner face of said second element 7 in view that those supplements 9 are placed coincidentally with specific zones of the foot, such as phalanges, metatarsals and the tarsal. It is important to emphasize that the geometrical conformation of the backing supplements 9 can adopt many forms, since the shape is mostly defined by the foot of the user.

To a better understanding of the present invention, the use of the foot correction device 1 will be described as well as the procedure to reeducate the global positioning of the foot using said device 1. Accordingly, the basic use of the device 1 comprises the steps of inserting the toes 6 within the gaps 4 defined between the separating means 3; slide said device 1 until the separating means 3 reaches the joints 20 of the toes 6; proceed with the adjustment of the separating means 3 with the variable adjustable means 10; continue with the adjustment of the fixing and adjusting means 8 from the second element 7 and the element 2 and optionally perform several specific routines.

Particularly, the device 1 can be used and defined as at least four different devices, such as an exercising device, and day foot correction device, a night foot correction device and an aquatic correcting device.

Exercising Foot Correction Device

This particular device it is made from fabrics and materials stress resistant so the user can perform several exercising activities wearing the device. The same is adjustable and comprises foot backing supplements (head of the 1^{st} to the 5^{th} metatarsus). The toe separating means are hard and anatomically configured. The device presents an anatomic design in order to meet the standards needed for sports and health.

The exercising device separates the toes y corrects the structural positioning of the foot aligning the bones, muscle, tendons and toe and metatarsus ligaments. It brings support and corrects front pressure points on the foot.

The basic function of the device is to allow the exercising of the foot from an anatomically correct position, stretches the muscles and tendons, generally invigorates the foot and specific muscles and generates mobility in the joints. The device also allows a correct walking posture by rebuilding the arcs and pressing points. Furthermore, it improves the different postures in several types of exercises such as Yoga, Tai Chi, Pilates and so.

The exercising device improves the global posture of the foot, strengthens muscles and tendons from the foot and ankle, reduces pains and cramps, generates greater vitality and agility to the foot by means of the own training and help rehabilitate the foot's arcs and the venous return.

Day Foot Correction Device

This device comprises soft toe separating means. It has a stretching adjust feature (it only covers the three middle toes). It also comprises an arc backing supplement. The day correction device allows correcting the posture of the toes and the metatarsus within the shoe. It also corrects the footstep and the pressure point, it helps rehabilitate feet diaphragm, improves the hip posture and the lumbar backbone correcting the active and passive posture of the foot.

The day correcting device also reshapes the bunion by changing the forces o tension lines and traction on the same. It also helps the venous return in the foot and leg, eases the pain and crams in foot and leg and it is great for persons who have to work standing up.

Night Foot Correction Device

This device comprises anatomic toe separating means made of soft and expandable material. It is made in a soft and breathable fabric and it is adjustable and washable. Its design is made for comfort.

The therapeutic action of the device allows correcting the toes and metatarsus positioning while the person is asleep.

Its main object is to adjust and correct the tension forces of the articulation on the 1^{st} toe and the 1^{st} metatarsus that provoke the Hallux Valgus or bunion specifically, and the rest of the metatarsus-phalange joints, improving the global posture of the foot in a passive mode and reducing the hammer toe or contracted toe syndrome. By changing the traction and tension forces on the bunion and on the rest of the joints, the device activates a physiological shaping process of the body. It also smoothly shapes the backing pressure points improving the posture of the metatarsus and the foot's arc in a passive way.

The associated benefits in the use of the night foot correction device are to generate the reshaping and reduction of the bunion. It also provides an optimum foot resting position while reducing the pain and night cram.

Aquatic Foot Correction Device

The corrective action of the device is to correct the position of the toes and metatarsals, allowing a better positioning of muscles, ligaments and joints on feet and legs while swimming. Their functions are to correct the position of bones, muscles, ligaments and joint of the foot and lower leg, so the forces pulling them are reduced when performing the muscle contraction during swimming. Supplemental support points also improve the position of the metatarsal bones and joints of the phalanges. On the other hand leads to greater resistance to water surface helping the propulsion in the water.

The benefits of aquatic corrector are to improve muscle cramps in the feet and legs, those caused by poor traction thereof. Improves muscle performance of feet and legs to undergo the effort and half the resistance of the water. Generates more surface water resistance and, therefore, enhances propulsion by the feet.

The device design includes aquatic waterproof adjustable means and also includes supplements of the foot (head of the 1st to the 5th metatarsal), toe separators presents anatomical and anatomical design relating to water sports. As seen from the described use of the device 1, the object of the present invention allows a global postural foot with supplements.

The overall objectives are to form healthy feet as well as provide a gradual training mode, easy and effective. Moreover, strengthening exercise and also allows dynamically feet from the correct anatomical position. Rehabilitate feet with structural problems, rearrange the skeletal structure, muscle and tendons of the foot, promotes joint mobility of the foot, restore points of the foot and ultimately prevent and resolve pain and cramps in legs and feet.

The following chart explains the procedure to be used to effectively treat sore feet, exostosis, hammer toes and high arches, lack of strength in the feet, ankles, legs, feet and legs with edema, nocturnal and day cramps.

CHART 1
CONTRACTION/STRETCHING AND STRETCHING/CONTRACTION
TIMES, REPETITIONS AND SERIES PER LEVEL

| | LEVEL | | |
|---|---|---|---|
| | BEGINNER | INTERMEDIATE | ADVANCED |
| REPETITIONS | 5 to 10 | 10 to 20 | 20 to 30 |
| TIME | None | 3 to 5 sec. | 5 to 15 sec. |
| MUSCULAR | | | |
| STRETCHING/ | | | |
| CONTRACTION | | | |
| TIME | 2 to 8 sec. | 8 to 15 sec. | 15 to 25 sec. |
| MUSCULAR | | | |
| CONTRACTION/ | | | |
| STRETCHING | | | |
| SERIES | 1-2 | 2-3 | 3-4 |

The benefits obtained by means of the routines shown on the above chart, are the following:
Strengthens y rearranges the bone and muscle structure of the foot.
Recovers joint mobility
Generates agile and light feet
Corrects bunions (hallux Valgus)
Strengthens y rearranges the bone and muscle structure of the legs
Eases backbone pains and/or hips (the ones produces by an incorrect posture and feet positioning)
Improves the blood stream in veins reducing varicose veins.
Improves ankles, uncured sprained ankles, knees and hips (changes the back support, muscle, joints by changing the position and traction forces).

1. A data processing apparatus, comprising:
a buffer configured to store interleaved data;
an interleaving index producing unit configured to produce an interleaving index of the interleaved data; and
an output control unit configured to output the interleaved data stored in the buffer using the interleaving index,
wherein the interleaving index producing unit is configured to produce the interleaving index using at least one logic block corresponding to a mathematical equation associated with a transmission mode of the interleaved data.

2. The data processing apparatus according to claim 1,
wherein the output control unit is configured to output the interleaved data stored in the buffer based on an order of production of the interleaving index.

3. The data processing apparatus according to claim 1,
wherein the buffer is configured to sequentially store the interleaved data based on an order of receiving the interleaved data.

4. The data processing apparatus according to claim 1,
wherein the interleaving index producing unit is configured to produce the interleaving index using at least one logic block corresponding to a mathematical equation for interleaving the interleaved data.

5. (canceled)

6. The data processing apparatus according to claim 1,
wherein the interleaving index producing unit comprises:
a mode determining unit configured to determine a transmission mode of the interleaved data;
a multiplier configured to perform a multiplication operation on a resulting value of a previous modular operation and an interleaving initial constant value;
an adder configured to perform an addition operation on a resulting value of the multiplication operation and an add-constant corresponding to the determined transmission mode;
a modular operating unit configured to perform a modular operation on a resulting value of the addition operation using a modular-constant corresponding to the determined transmission mode;
a modular operation determining unit configured to determine whether a resulting value of the modular operation corresponds to an allowable range of the determined transmission mode;
a subtractor configured to perform a subtraction operation on the resulting value of the modular operation and a subtract-constant corresponding to the determined transmission mode when determined that the resulting value of modular operation corresponds to the allowable range of the determined transmission mode; and
a feedback unit configured to feedback the resulting value of modular operation to the multiplier.

7. The data processing apparatus according to claim 6,
wherein the modular operating unit is configured to perform a modular operation on the resulting value of addition operation using a bit equal to or lower than the modular-constant of the resulting value of the addition operation.

8. The data processing apparatus according to claim 1,
wherein an operating frequency for producing the interleaving index is set higher than an operating frequency of the data output.

9. An interleaving index producing apparatus configured to:
produce an interleaving index of an interleaved data used in de-interleaving output control of the interleaved data stored in a predetermined buffer memory; and
wherein the interleaving index producing apparatus is configured to produce the interleaving index using at least one logic block corresponding to a mathematical equation for interleaving the interleaved data,
wherein the interleaving index producing unit includes a mode determining unit configured to determine a transmission mode of the interleaved data.

10. The interleaving index producing apparatus according to claim 9,
wherein the interleaving index producing unit includes:
a multiplier configured to perform a multiplication operation on a resulting value of a previous modular operation and an interleaving initial constant value;
an adder configured to perform an addition operation on a resulting value of the multiplication operation and an add constant corresponding to the determined transmission mode;
a modular operating unit configured to perform a modular operation on a resulting value of the addition operation using a modular constant corresponding to the determined transmission mode;
a modular operation determining unit configured to determine whether a resulting value of the modular operation is within an allowable range of the determined transmission mode;
a subtractor configured to perform a subtraction operation on the resulting value of modular operation and a subtract constant corresponding to the determined transmission mode when the resulting value of modular operation is within an allowable range of the determined transmission mode; and
a feedback unit configured to feedback the resulting value of modular operation to the multiplier.

11. A data processing method, comprising:
storing interleaved data;
producing an interleaving index of the interleaved data; and
outputting the stored interleaved data using the produced interleaving index,
wherein producing the interleaving index comprises producing the interleaving index using at least one logic block corresponding to a mathematical equation defined for each transmission mode of the interleaved data.

12. The data processing method according to claim 11,
wherein outputting comprises outputting the stored interleaved data based on an order of producing the interleaving index.

13. The data processing method according to claim 11,
wherein storing the interleaved data comprises sequentially storing the interleaved data based on an order of receiving the interleaved data.

14. The data processing method according to claim 11,
wherein producing the interleaving index comprises producing the interleaving index using at least one logic block corresponding to a mathematical equation for interleaving the interleaved data.

15. (canceled)

16. The data processing method according to claim 11,
wherein producing the interleaving index comprises:
determining a transmission mode of the interleaved data;
performing a multiplication operation on a resulting value of a previous modular operation and an interleaving initial constant value;
performing an addition operation on a resulting value of the multiplication operation and an add constant corresponding to the determined transmission mode;
performing a modular operation on a resulting value of the addition operation using a modular constant corresponding to the determined transmission mode;
determining whether a resulting value of the modular operation is within an allowable range of the determined transmission mode;
performing a subtraction operation on the resulting value of the modular operation and a subtract constant corresponding to the determined transmission mode when the resulting value of the modular operation is determined to be within the allowable range of the determined transmission mode; and
feedbacking the resulting value of the modular operation to the multiplier.

17. The data processing method according to claim 16,
wherein performing the modular operating comprises performing a modular operation using a bit equal to or lower than the modular constant of the resulting value of the addition operation.

18. The data processing method according to claim 11,
further comprising setting an operating frequency for producing the interleaving index at a higher level than an operating frequency of the data output.

19. An interleaving index producing method comprising:
producing an interleaving index of an interleaved data used in de-interleaving output control of the interleaved data stored in a predetermined buffer memory,
wherein the interleaving index is produced using at least one logic block corresponding to a mathematical equation for interleaving the interleaved data,
wherein producing the interleaving index comprises determining a transmission mode of the interleaved data.

20. The interleaving index producing method according to claim 19,
wherein producing the interleaving index comprises:
performing a multiplication operation on a resulting value of a previous modular operation and an interleaving initial interleaving constant value;
performing an addition operation on a resulting value of the multiplication operation and an add constant corresponding to the determined transmission mode;
performing a modular operation on a resulting value of the addition operation using a modular constant corresponding to the determined transmission mode;
determining whether a resulting value of the modular operation is within an allowable range of the determined transmission mode;
performing a subtraction operation on the resulting value of modular operation and a subtract constant corresponding to the determined transmission mode when the resulting value of modular operation is determined to be within the allowable range of the determined transmission mode; and
feedbacking the resulting value of the modular operation to the multiplier.

21. A computer readable medium embodying instructions to cause a data processing apparatus to perform de-interleaving comprising:
storing interleaved data;
producing an interleaving index of the interleaved data; and
outputting the stored data using the produced interleaving index,
wherein producing the interleaving index comprises producing the interleaving index using at least one logic block corresponding to a mathematical equation defined for each transmission mode of the interleaved data.
